(19) United States
(12) Patent Application Publication
(10) Pub. No.: US 2024/0382186 A1
(43) Pub. Date: Nov. 21, 2024

(54) TONGUE BASE SAMPLING INSTRUMENT

(71) Applicant: Sentry Endoscopy Ltd., Jerusalem (IL)

(72) Inventors: Mark Rutenberg, Monsey, NY (US); Angel Mendoza, Tamarac, FL (US)

(21) Appl. No.: 18/669,511

(22) Filed: May 20, 2024

Related U.S. Application Data
(60) Provisional application No. 63/467,724, filed on May 19, 2023.

(51) Int. Cl. A61B 10/02; A61B 1/018; A61B 1/05; A61B 1/06; A61B 1/24; A61B 10/04

(52) U.S. Cl. CPC A61B 10/02; A61B 1/018; A61B 1/05; A61B 1/0676; A61B 1/24; A61B 10/04; A61B 2010/0216

(57) ABSTRACT

A handheld intra oral instrument having a channel through which a sampling brush is threaded. The distal end of the tube is bent at greater than 100 degrees allowing imaging of and contact with tissue areas such as the vallecula. The terminal end of the instrument is provided with a video chip and a light source to illuminate the desired area and provide real time video images.

### RELATED APPLICATIONS

This application claims the benefit of pending Provisional Appl. 63/467,724 filed May 19, 2022, the contents of which are incorporated by reference herein.

### FIELD OF THE INVENTION

The invention relates to cell and tissue sampling devices, more specifically to a device configured to observe and sample the base of tongue and oropharyngeal regions.

### BACKGROUND OF THE INVENTION

Head and neck cancers very often emanate from the mucosal epithelium in the oral cavity, pharynx and larynx. These cancers are widely known as head and neck squamous cell carcinoma (HNSCC). HNSCC's are often detected as cancers, yet such cancers can be prevented if the disease was caught in the dysplastic stages. There is therefore a need in the art for an instrument that can view and sample tissue of the base of tongue.

### SUMMARY OF THE INVENTION

Embodiments of the invention provide a handheld intra oral instrument having a channel through which a sampling brush is threaded. The distal end of the tube is provided with a video chip and a light source to illuminate the desired area and provide real time video images. The instrument is angled in a manner to reach heretofore unreachable tissue areas, such as for example the vallecula.

Embodiments of the invention further provide a method of visualizing a base of tongue and vallecula area and determining a tissue area that requires sampling. The invention further includes the step of moving a circular brush head out of a stored position to a deployed position and sampling the desired tissue area using the brush head.

In embodiments of the invention, control hardware and/or software (e.g. PCB and power converter etc.) are provided in a separate device apart from the sampling device, thereby allowing the sampling device to be relatively inexpensive and suitable for single use.

### BRIEF DESCRIPTION OF THE DRAWINGS

Illustrative embodiments of the invention will hereafter be described with reference to the accompanying drawings, wherein like numerals denote like elements.

FIG. 1 is a side view of a sampling device in accordance with an embodiment of the invention.

FIG. 2 is an enlarged side view of a terminal end of a sampling device in accordance with an embodiment of the invention.

FIG. 3 is an elevational view of the terminal end of a sampling device in accordance with an embodiment of the invention.

FIG. 4 is an elevational view of the terminal end of a sampling device shown partially broken away in accordance with an embodiment of the invention.

FIG. 5 is perspective view of terminal end of a sampling device with a brush head shown in schematic view in accordance with an embodiment of the invention.

FIG. 6 is a side cross-sectional view of a sampling device and connected wire in accordance with an embodiment of the invention.

FIG. 7 terminal is a cross-sectional schematic view of a sampling device shown in use in accordance with an embodiment of the invention.

### DETAILED DESCRIPTION OF THE INVENTION

Embodiments of the present invention will now be described with reference to the above-identified figures of the drawings. However, the drawings and the description herein of the invention are not intended to limit the scope of the invention. It will be understood that various modifications of the present description of the invention are possible without departing from the spirit of the invention. Also, features described herein may be omitted, additional features may be included, and/or features described herein may be combined in a manner different from the specific combinations recited herein, all without departing from the spirit of the invention.

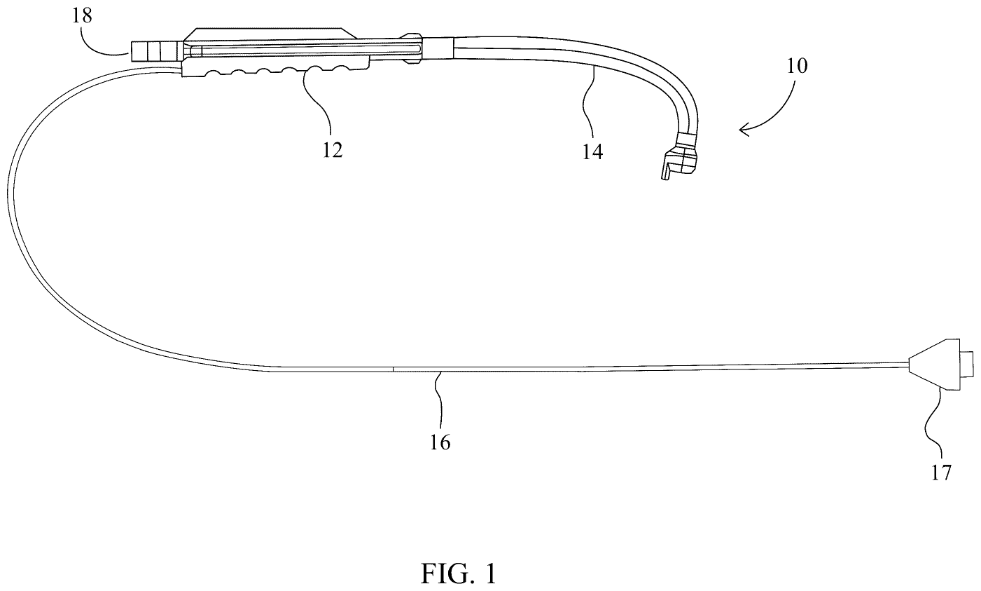

FIG. 1 shows a tissue sampling device 10 in accordance with an embodiment of the invention. As shown, sampling device 10 has a handle 12 with a tube 14 extending therefrom. A first side of handle 12 has an electrical wire 16 extending therefrom. Wire 16 has a terminal end that is provided with a connector 17 for connecting to a video processing drive or similar control device.

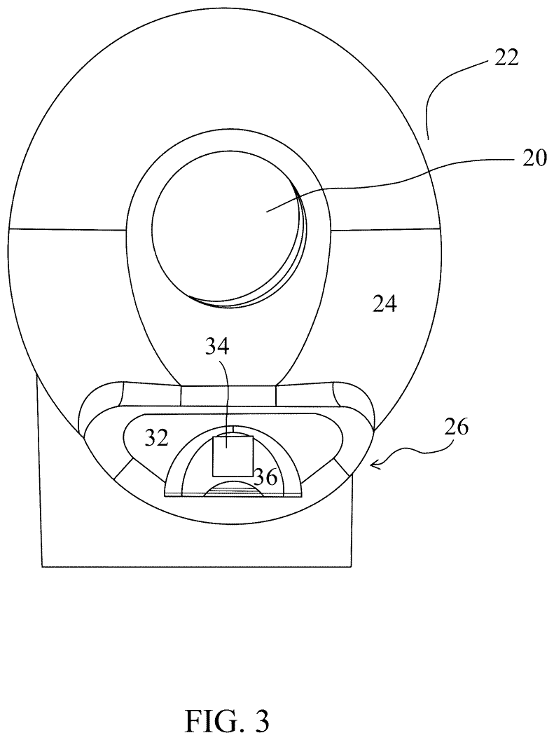

Tube 14 has a channel extending therethrough. For example, as shown, a first tube opening 18 is shown in FIG. 1. With reference to FIG. 3, a second tube opening 20 is shown at the distal end of the tube 14.

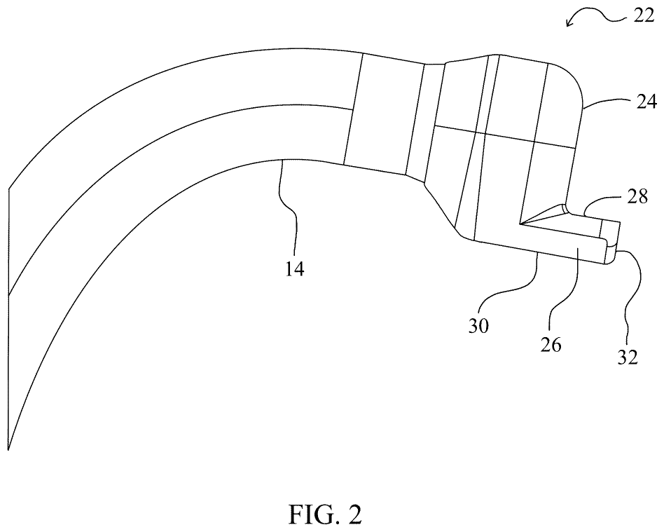

As shown in FIG. 2, distal end 22 of tube 14 is shown having a substantially flat end 24 and a lower extending segment 26. Extending segment 26 has an upper surface 28, an underside surface 30 and an end surface 32. In a preferred embodiment of the invention, extending segment 26 is disposed at a substantial right angle to flat end 24.

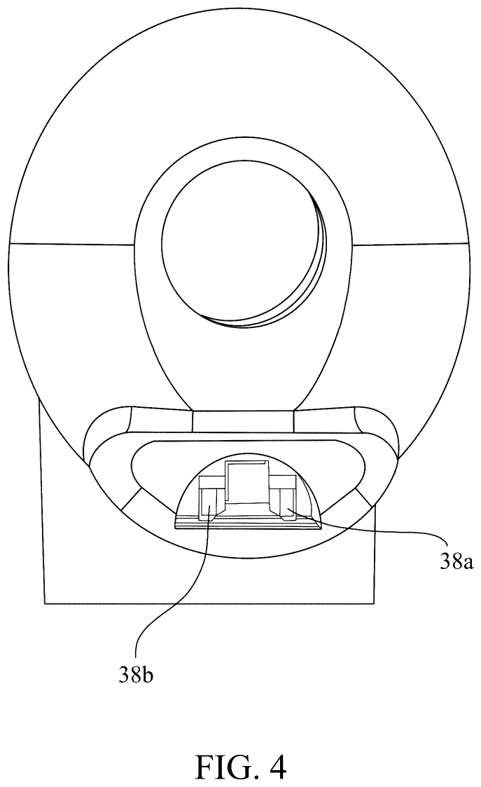

With reference to FIG. 3, a video chip 34 or similar image capturing device is positioned at a central region of end surface 32 of extending segment 26. Video chip 34 is used to deliver real time images to a monitor or similar display. One or more LEDs or similar light-emitting devices are provided for illuminating a desired tissue area. In an embodiment of invention an LED is provided on each side of video chip 34. For example, as best shown in the cut-away view of FIG. 4, a first LED 38a is provided on a right side of video chip 34 and a second LED 38b is provided on a left side of video chip 34. In a preferred embodiment, video chip 34 and LEDs 38a, 38b are covered by a plastic transparent window 36 (as shown in FIG. 3) or are similarly encapsulated.

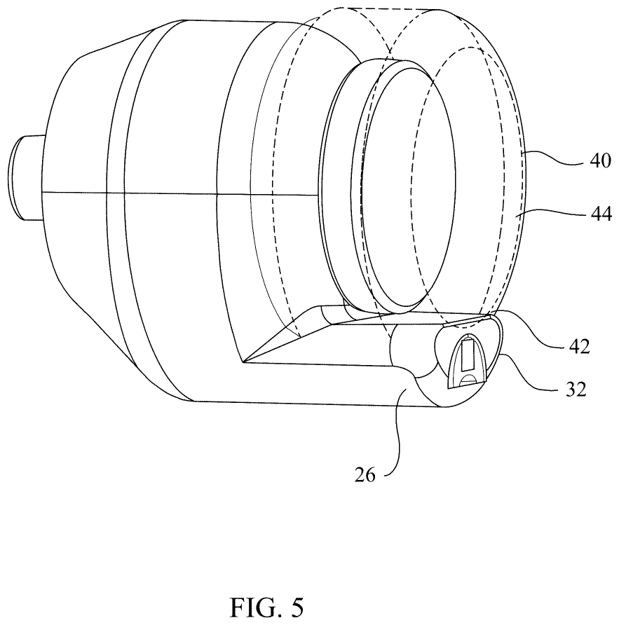

In an embodiment of the invention, distal end of tube 14 is configured to retain a round brush head. With reference to the schematic illustration in FIG. 5, a toroidal head 40 is shown approximating the outside surface of a round brush head. A lower aspect 42 of brush head 40 rests on or near upper surface 28 of extending segment 26. A front aspect 44 of brush head 40 is disposed substantially coplanar with end surface 32 of extending segment 26.

Brush head 40 extends from a flexible handle (not shown). The flexible handle is threaded through the channel of the sampling device 10. The handle extends out of first channel opening 18 thereby providing a user with ability to grasp the handle in order to control the brush head 40.

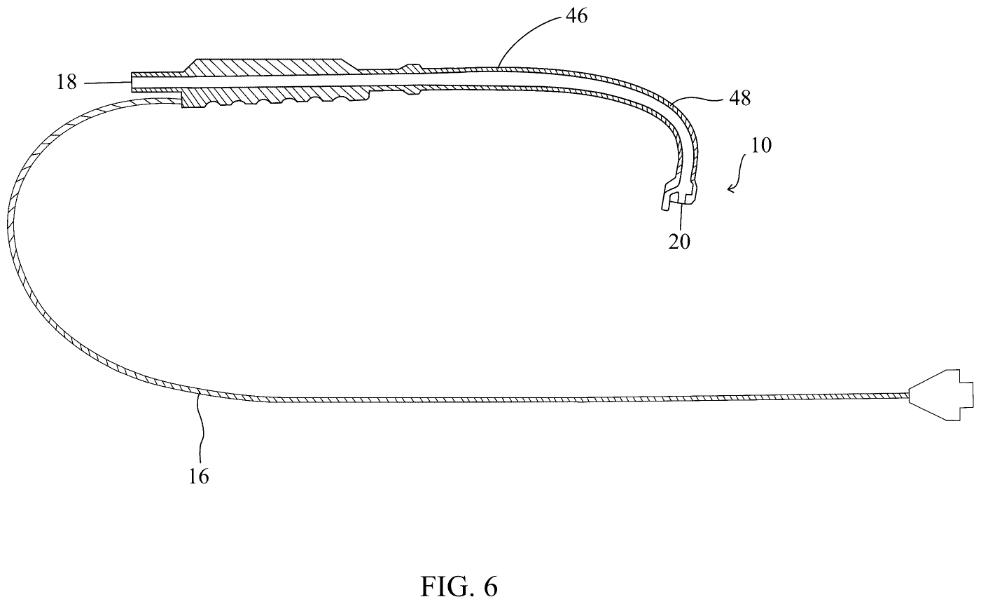

FIG. 6 shows a cross-sectional view through sampling device 10. A channel 46 is shown extending the length of device 10 between first opening 18 and second opening 20.

It will be understood that wire 16 is configured to deliver electrical power to the video chip 34 and LEDs 38a, 38b and to deliver a live video feed to a monitor or similar display.

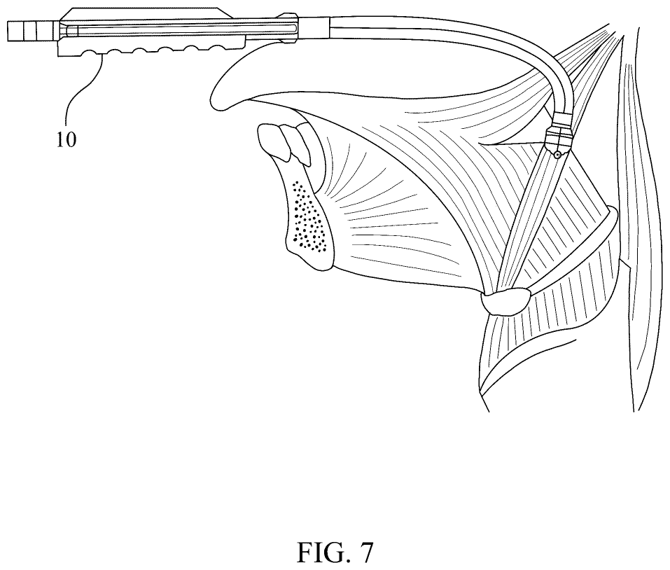

With continued reference to FIG. 6, a bend 48 is made at a terminal aspect of device 10. In embodiments of the invention, bend 48 is approximately 100° to 110°. In one embodiment, the bend is approximately 110°. In embodiments of the invention, device 10 so configured may be used to image and sample regions at the base of the tongue such as the vallecula, the epiglottis or neighboring tissue areas. For example, with reference to the schematic view shown in FIG. 7, device 10 is shown deployed in a patient's oral cavity with terminal end of device 10 turned downward and slightly inward thereby reaching the areas behind the and below the base of the patient's tongue.

In use, a physician deploys a device 10 by inserting a brush into the channel such that the brush head is maintained in the space above of extending segment 26. With brush head so positioned, the light emitting elements and video capturing device provided on extending segment 26 remain unobstructed by the brush head and allow a clinician to clearly see tissue and anatomical structures in the base of tongue, larynx and hypopharynx. Once a tissue area of interest is identified, the clinician pushes the brush handle in order to urge the brush head forward to contact the desired area. The clinician may also rotate the brush's handle to collect a cellular or tissue sample. For example, brush head 40 may be moved forward past extending segment 26. In this “deployed” position, brush head 40 may contact tissue for collection of cells.

It will be understood that any of various biopsy brushes may be used in embodiments of the invention. For example, in one embodiment of the invention, a toroidal brush similar to the brush head that is described in U.S. Pat. No. 6,258,044, the disclosure of which is incorporated by reference herein.

While this invention has been described in conjunction with the embodiments outlined above, it is evident that many alternatives, modifications and variations will be apparent to those skilled in the art. Accordingly, the exemplary embodiments of the invention, as set forth above, are intended to be illustrative, not limiting. Various changes may be made without departing from the spirit and scope of the invention.

What is claimed is:

1. A tissue sampling device, comprising:
a tubular member having a first end and second end;
a handle disposed on a first end of the tubular member;
a channel disposed through a portion of the tubular member;
the tubular member comprising a bend of greater than 100°; and
an image capturing device and a light source provided on the second end of the tubular member.

2. The tissue sampling device of claim 1 whereby the bend is between approximately 100° to 110°.

3. The tissue sampling device of claim 1, whereby the second end of the tubular member comprises a substantially flat end.

4. The tissue sampling device of claim 3, whereby the second end further comprises a lower extending segment that extends past the flat end.

5. The tissue sampling device of claim 4, whereby the lower extending segment comprises an upper surface, an underside surface and an end surface.

6. The tissue sampling device of claim 5, upper surface of the extending segment is substantially orthogonal to the flat end of the second end of the tubular member.

7. The tissue sampling device of claim 6, whereby the end surface of extending segment comprises a video chip.

8. The tissue sampling device of claim 7, whereby the whereby the end surface of extending segment comprises a light source.
